(19) United States
(12) Patent Application Publication
(10) Pub. No.: US 2021/0361706 A1
(43) Pub. Date: Nov. 25, 2021

(54) COMBINATION OF NATURAL KILLER CELLS WITH CYCLOPHOSPHAMIDE COMPOUNDS FOR THE TREATMENT OF CANCER

(71) Applicant: ACADEMIA SINICA, Taipei (TW)

(72) Inventors: Nan-Shih LIAO, Taipei (TW); Shih-Wen HUANG, Taipei (TW); Zhen-Qi WU, Taipei (TW); Yein-Gei LAI, Taipei (TW); Yae-Huei LIOU, Taipei (TW)

(73) Assignee: ACADEMIA SINICA, Taipei (TW)

(21) Appl. No.: 17/269,612

(22) PCT Filed: Aug. 28, 2019
(86) PCT No.: PCT/US2019/048498

§ 371 Date: Feb. 19, 2021

Related U.S. Application Data
(60) Provisional application No. 62/724,338, filed on Aug. 29, 2018.

(51) Int. Cl. A61K 35/17; A61K 31/675; A61P 35/00

(52) U.S. Cl. CPC A61K 35/17; A61P 35/00; A61K 31/675

(57) ABSTRACT

Combined therapy of cancer (e.g., breast cancer) involving a cyclophosphamide compound and natural killer (NK) cells. Also provided herein are methods for inducing immune memory and/or reducing the risk of tumor recurrence using the combined therapy of a cyclophosphamide compound and NK cells.

### CROSS REFERENCE TO RELATED APPLICATIONS

This application claims the benefit of the filing dates of U.S. Provisional Application No. 62/724,338, filed Aug. 29, 2018, the entire contents of which is incorporated by reference herein.

### BACKGROUND OF INVENTION

Natural killer (NK) cells play a critical role in anti-tumor immunity. Human and mice with impaired NK cell development or function show increased tumor susceptibility, while mice with defective intrinsic negative regulation of NK cells show augmented anti-cancer activity. Imai et al., Lancet, 356(9244):1795-1799 (2000); Paolino et al., Nature, 507(7493):508-512 (2014); and Waldhauer et al., Oncogene, 27(45):5932-5943 (2008). The levels of IFN-γ production and intra-tumor NK cell were found to be positively associated with survival of patients with gastrointestinal stromal tumors. Menard et al., Cancer Res, 69(8):3563-3569 (2009); Rusakiewicz et al., Cancer Res, 73(12):3499-3510 (2013); and Waldmann et al., J Investig Dermatol Symp Proc, 16(1):528-30 (2013). The increased expression of molecules related to NK cell activation in breast cancer also associates with favorable prognosis. Ascierto et al., J Transl Med., 12; 11:145 (2013).

NK cell can recognize cancer cell through NK receptors, which results in NK cell activation. Vivier et al., Science, 331(6013):44-49 (2011). The activated NK cells not only kill the tumor cells directly by perforin/gramzyme, TRAIL, or Fas ligand, but also produce cytokines and chemokines to induce type 1 immune response (e.g., including induction of TH1 CD8⁺ cytotoxic T cell, and type 1 macrophages), thereby orchestrating effective anti-tumor immunity. Mocikat et al., Immunity, 19(4):561-569 (2003). Among the NK cell-produced factors, IFN-γ is the prominent one to induce type 1 T cell and macrophage responses. These effector functions of NK cells take place promptly upon recognition of tumor cells without the need of prior activation.

Given the prominent role of NK cell in anti-tumor immunity, adoptive NK cell transfer has been tested for cancer immunotherapy (Guillerey et al., Nature immunology 2016, 17(9):1025-1036) and found mainly effective in the treatment of certain myeloid leukemia under allogeneic setting Geller et al., Immunotherapy 2011, 3(12):1445-1459; and Morena et al., L, Blood 2011, 117(3):764-771). On the other hand, transfer of autologous NK cells showed little clinical benefit in treating solid tumors. Burns et al., Bone marrow transplantation, 32(2):177-186 (2003); Parkhurst et al., Clin Cancer Res, 17(19):6287-6297 (2011); and Sakamoto et al., Journal of translational medicine, 13:277 (2015).

It is therefore of great interest to develop suitable treatment regimens to enhance the efficacy of adoptive NK cell transfer therapy.

### SUMMARY OF INVENTION

The present disclosure is based, at least in part, on the unexpected discoveries that a cyclophosphamide (CTX) compound enhanced the efficacy of natural killer (NK) cells against tumor growth and/or recurrence as observed in a syngeneic orthotopic mouse model of breast cancer. Surprisingly, combined treatment of the CTX compound and NK cells improved the long-term survival of tumor-bearing mice and tumor-resected mice compared to either CTX monotherapy or NK cell monotherapy. Furthermore, treatment with the combination induced immune memory that protected re-challenge with the same tumor, implying prevention of recurrence.

Accordingly, one aspect of the present disclosure provides a method of treating cancer, comprising: (a) administering to a subject in need thereof an effective amount of a cyclophosphamide (CTX) compound (e.g., CTX, mafosfamide, ifosfamide, or trofosfamide, or a pharmaceutically acceptable salt thereof); and (b) administering to the subject an effective amount of NK cells. In some embodiments, the NK cells may have been exposed to IL-15 and/or IL-12 ex vivo prior to step (b).

In some instances, step (a) may comprise at least two doses of the CTX compound. The two consecutive doses of the CTX compound may be administered to the subject 4-8 days apart, for example, 6 days apart. Alternatively or in addition, step (b) may comprise at least two doses of the NK cells.

In some embodiments, at least one dose of the CTX compound in step (a) can be administered to the subject before step (b). In some examples, all doses of the CTX compound in step (a) may be administered to the subject before step (b). Alternatively, the subject may be given the CTX compound before and after administration of the NK cells.

In other embodiments, at least one dose of the NK cells in step (b) can be administered to the subject before step (a). In some examples, all doses of the NK cells in step (b) may be administered to the subject before step (a). Alternatively, the subject may be given the NK cells before and after administration of the CTX compound.

In some embodiments, the subject is human patient having, suspected of having, or at risk for cancer, for example, a solid cancer or a hematologic cancer. Exemplary solid cancers include, but are not limited to breast cancer, prostate cancer, liver cancer, lung cancer, melanoma, pancreatic cancer, or bladder cancer. In one example, the solid cancer is breast cancer. Exemplary hematologic cancer includes leukemia, lymphoma, or multiple myeloma. In some embodiments, the subject has had a tumor resection.

In some embodiments, the effective amount of the CTX compound and the effective amount of NK cells, in combination, are effective in reducing the risk of cancer recurrence.

In another aspect, the present disclosure provides a method of inducing protective immune memory against tumor recurrence and/or reducing the risk of tumor recurrence, the method comprising: (a) administering to a subject in need thereof an effective amount of a cyclophosphamide (CTX) compound such as CTX; and (b) administering to a subject an effective amount of NK cells. In some examples, the subject is a human patient who had tumor. Such a tumor patient may have undergone an anti-tumor therapy.

In any of the methods disclosed herein, the NK cells can be autologous. Alternatively, the NK cells may be allogenic. In other embodiments, the NK cells can be from an NK cell line, derived from pluripotent stem cells, or derived from induced-pluripotent stem cells.

Further, provided herein is a kit for use in treating cancer, comprising (i) a cyclophosphamide (CTX) compound such as CTX; and (ii) NK cells. In some embodiments, the NK cells have been exposed to IL-15 and IL-12 ex vivo.

Also with the scope of the present disclosure are pharmaceutical compositions for use in treating cancer (e.g., those described herein), wherein the pharmaceutical compositions comprise a CTX compound as disclosed herein and NK cells as also disclosed herein (e.g., formulated together or separately), as well as uses of the CTX compound and the NK cells for manufacturing a medicament for cancer therapy.

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

FIGS. 1A-1B is a series of graphs showing the therapeutic effect of cyclophosphamide (CTX) treatment on EO771 tumor-bearing mice. FIG. 1A shows the survival of EO771 tumor-bearing mice treated with once or twice CTX at 150 mg/kg per injection. FIG. 1B shows the survival of EO771 tumor-bearing mice treated with twice CTX at a dosage of 125 or 150 mg/kg per injection. Mice in each treatment group were compiled from two to six independent experiments. ****p≤0.0001 by Log-rank test.

FIG. 2 is a graph showing that CTX and NK cell therapies synergistically enhance the survival of EO771 tumor-bearing mice. EO771 tumor-bearing mice were treated with PBS, NK cells, CTX, or CTX plus NK cells starting from day-21 post tumor inoculation and monitored for survival. Data in each group were compiled from two to six independent experiments. ****p≤0.0001 by Log-rank test.

FIG. 3 is a graph showing that CTX and NK cell combination therapy induces immune memory in EO771 tumor-bearing mice. Tumor-bearing mice survived the primary EO771 tumor after treatment were re-challenged with EO771 cells, and then analyzed for survival. Age-matched naïve mice were inoculated with EO771 cells the first time to serve as the control group. Data were compiled from 2 to 5 independent experiments. ****p≤0.0001 by Log-rank test.

FIG. 4 is a graph showing that CTX and CTX plus NK cell therapies achieve comparable efficacy in the survival of EO771 tumor-resected mice. Mice were orthotopically inoculated with EO771 cells. One group of mice received no treatment (PBS group). Other mice received tumor and draining lymph node resection 21 days later. The tumor-resected mice either received no further therapy (resection only) or treated with CTX or/and NK cells. All groups of mice were subjected to Kaplan-Meier survival analysis. Data of each group were compiled from two to seven independent experiments. ****p≤0.0001 by Log-rank test.

FIG. 5 is a graph showing that NK cell treatment potentiates tumor-specific immune memory in EO771 tumor-resected mice. Tumor-resected mice survived the primary EO771 tumor after treatment were re-challenged with EO771 cells, and then analyzed for survival. Age-matched naïve mice were inoculated with EO771 cells the first time to serve as the control group. Data were compiled from 2 to 5 independent experiments. ****p≤0.0001 and **p≤0.01 by Log-rank test.

### DETAILED DESCRIPTION OF INVENTION

The present disclosure is based, at least in part, on the unexpected discovery that the combined therapy of a cyclophosphamide (CTX) compound and natural killer (NK) cells eradicated tumors as exemplified in a syngeneic orthotopic animal model of breast cancer and induced immune protection against tumor recurrence in approximately 75% of mice under either tumor-bearing or tumor-resected conditions.

Although NK cells play critical role in anti-tumor immunity, adoptive transfer of NK cells had shown limited clinical benefit except for certain myeloid leukemia under allogeneic setting. A possible reason is that the tumor microenvironment (TME) suppresses the anti-tumor activity of NK cells. Indeed, tumor cells and the intra-tumor myeloid cells, stromal cells, and endothelial cells produce suppressive factors that inhibit NK cell function. Hasmim et al., Front Immunol, 6:482 (2015). Therefore, developing methods to harness NK cell anti-tumor activity in the immune suppressive TME would be of significance for developing effective cancer immunotherapy.

Provided herein, in some embodiments, are methods and kits that address the limitation. The methods disclosed herein involve the combined use of NK cells and a cyclophosphamide (CTX) compound in cancer treatment. The experimental data provided herein suggest that the CTX compound could reduce immune suppression in TME, thereby facilitating the anti-tumor effects of the NK cells. Without being bound by a particular theory, the CTX compound may not only induce immunogenic death of cancer cell, but also ablates immunosuppressive immune cell and induces bacteria translocation from the gut into secondary lymphoid tissues, which may enhance anti-tumor immune response. Therefore, the newly developed immune cells have the opportunity to generate effective anti-tumor response in a much less immunosuppressive TME under the influence of transferred NK cells that drive type 1 immune response via production of IFN-γ.

As described below, the therapeutic effect can be evaluated at two levels, the survival from primary tumor and the survival from re-challenge with the same tumor cells. The latter represents acquisition of immune memory that prevents or reduces the risk of tumor recurrence. An estimated cure rate can be obtained by multiplication of the two survival rates. NK cell monotherapy showed little to low efficacy and CTX monotherapy showed higher efficacy as compared to the NK cell therapy. Surprisingly, the combined therapy of the NK cell and the CTX compound resulted in long-term survival with protective immune memory in approximately 75% of tumor-bearing and tumor-resected mice. The estimated cure rate of the combined therapy is higher than either mono-therapy. EO771 breast cancer cell line carries p. 53 mutation, and spontaneously metastasizes. Johnstone et al., Disease models & mechanisms, 8(3):237-251 (2015); and Ewens et al., Anticancer research, 25(6b):3905-3915 (2005). As shown herein, the NK cell and CTX combined therapy is efficacious in this aggressive breast cancer model.

Accordingly, described herein are natural killer (NK) cells and cyclophosphamide compounds, for example, for use in treating cancer and/or reducing the risk of cancer recurrence and kits comprising the NK cells and cyclophosphamide compounds.

Natural Killer (NK) Cells

One aspect of the present disclosure provides natural killer (NK) cells and cyclophosphamide (or derivatives thereof) for use, for example, in adoptive cell transfer therapy.

NK cells are cytotoxic lymphocytes that have been implicated in innate immunity and may be characterized by their ability to eradicate target cells (e.g., tumor cells, virally infected cells, and bacterial cells) without prior activation. Suitable NK cells may be derived from any species, including mammals (e.g., humans, mouse, rat, dog, and sheep). When used for adoptive transfer therapy to a subject, the source of NK cells may be chosen to minimize induction of an inflammatory response (e.g., host versus graft disease). For example, the NK cells may be autologous, i.e., derived from the same subject to whom the NK cells are to be administered. Alternatively, the NK cells may be allogenic, e.g., derived from a donor of the same species and share the same HLA type as the subject who will be treated by the NK cells. In some embodiments, the NK cells are human NK cells derived from a human donor whose HLA antigens are acceptable matches to the subject to be treated by the NK cells.

Alternatively, the NK cells may be derived from a cell line (e.g., NK-92). In some embodiments, the NK cells may have antigen presenting cell (APC) properties and/or functions. Any of the NK cells may be derived from pluripotent stem cells. Alternatively, the NK cells may be derived from induced-pluripotent stem cells.

The NK cells of the present disclosure may also be expanded ex vivo (e.g., prior to administration to a subject). In some instances, the NK cells are exposed to a cytokine, including but not limited to IL-15 and IL-12. NK cells may be exposed to at least one (e.g., at least two, at least three, at least four, at least five, or at least 10) cytokine. In cases in which two or more cytokines are used, the cytokines may be used sequentially (in any order) or simultaneously. An exemplary method of culturing NK cells ex vivo is provided in Example 6.

For example, the NK cells may be expanded and activated with IL-15 and IL-12 ex vivo, following conventional methods. See, e.g., Fehniger et al., Journal of immunology, 162(8):4511-4520 (1999). IL-15 supports NK cell growth and survival, while both IL-15 and IL-12 stimulate the type 1 function of NK cells by up-regulating cytotoxicity and IFN-γ production. Without being bound by a particular theory, the ex vivo expansion of fresh NK cells in these two cytokines likely synchronize NK cell function toward a type 1 immune response driver.

IL-15 is a member of the 4-alpha-helix bundle family of cytokines and has been implicated promoting differentiation and proliferation of B cells, T cells and natural killer cells (see, e.g., Mishra et al., Clin Cancer Res., 20(8):2044-50, 2014). As an example, the amino acid sequence of human IL-15 is provided in GenBank Accession No. NP_751915.1.

IL-12 is a member of the interleukin 12 family of cytokines, which includes IL-12, IL-23, IL-27 and IL-35. This cytokine is made of two subunits. As a heterodimer, IL-12 is composed of IL-12 subunit alpha (IL-12 p35) and IL-12 subunit beta (IL-12 p40). In humans, each subunit is encoded by a different gene. For example, human IL-12 subunit alpha (e.g., GenBank Accession Numbers NP 000873.2, NP 001341511.1 and NP 001341512.1) is encoded by the IL12A gene, while human IL-12 subunit beta (e.g., GenBank Accession Number NP 002178.2) is encoded by the IL12B gene.

Cyclophosphamide (CTX) Compounds

As described herein, cyclophosphamide (CTX) compounds (e.g., CTX, pharmaceutically acceptable salts or esters thereof, or derivatives thereof) may be used to enhance the efficacy of NK cells in adoptive cell transfer therapy. Cyclophosphamide compounds are DNA cross-linking agent and can be metabolized into phosphoramide mustard. Structurally, cyclophosphamide compounds comprise a core structure of Formula I:

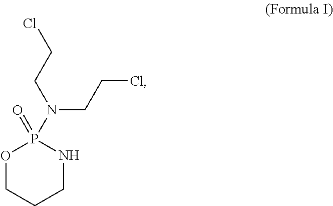 [SMILES: O=P1(N(CCCl)CCCl)NCCCO1]

which may optionally be substituted

It should be appreciated that cyclophosphamide derivatives (e.g., preactivated cyclophosphamide analogs) and pharmaceutically acceptable salts or esters thereof are also encompassed by the present disclosure. For example, one or more positions in Formula I may be modified (e.g., through substitution or addition of a functional group). Non-limiting examples of functional groups include hydrocarbons chains (e.g., substituted or unsubstituted alkyl, alkenyl, or alkynyl groups), benzene rings, amine groups, alcohols, ethers, alkyl halides, thiols, aldehydes, ketones, esters, carboxylic acids, and amides. For example, in Formula I, any of the carbons may be modified with a functional group, the hydrogen in the amine group may be substituted with a functional group, and chlorine may also be substituted with any halogen, including, but not limited to fluorine and iodine. Cyclophosphamide, pharmaceutically acceptable salts, and derivatives thereof may be synthesized using routine methods. See, e.g., Takamizawa et al., J Med Chem. 1975 April; 18(4):376-83. Exemplary cyclophosphamide derivatives include mafosfamide, ifosfamide, and trofosfamide.

The CTX compounds described herein, where applicable can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

In some examples, the CTX compound used in the methods disclosed herein may be an (R)-isomer. Alternatively, the CTX compound may be an (S)-isomer. In some examples, the CTX compound may be a mixture of (R) and (S) isomers.

The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

Pharmaceutical Compositions

In some embodiments, the present disclosure provides pharmaceutical compositions comprising a CTX compound (e.g., CTX) as disclosed herein, and/or the NK cells as also disclosed herein, together with a pharmaceutically acceptable carrier, diluent or excipient.

A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the CTX compound and/or the NK cells) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

A pharmaceutical composition comprising any of the CTX compounds and/or NK cells described herein may be administered by any administration route known in the art, such as parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration.

In some embodiments, the pharmaceutical compositions or formulations are for parenteral administration, such as intravenous, intra-arterial, intra-muscular, subcutaneous, or intraperitoneal administration. In some embodiments, compositions comprising NK cells can be formulated for intravenous infusion.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

In some embodiments, the pharmaceutical compositions or formulations comprising a CTX compound are suitable for topical administration to a subject. The inhibitor may be applied topically in the form of a lotion, solution, cream, ointment or dusting powder, or may be transdermally administered, for example, by the use of a skin patch. For application topically to the skin, the inhibitor can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Formulations suitable for topical administration in the mouth include lozenges comprising the active ingredient; pastilles comprising the active ingredient in an inert basis such as gelatin and glycerin, or sucrose and acacia; and mouth-washes comprising the active ingredient in a suitable liquid carrier.

The formulations may be presented in unit-dose or multi-dose containers, for example sealed ampoules or vials, and may be stored in a freeze-dried (lyophilized) condition requiring only the addition of the sterile liquid carrier immediately prior to use.

Therapeutic Applications

The present disclosure also provides combined therapy for cancer involving both a CTX compound such as CTX and NK cells as disclosed herein. The CTX compound and the NK cells may be administered simultaneously or sequentially (in any order) to a subject in need of the treatment.

To practice the therapeutic methods described herein, an effective amount of a cyclophosphamide compound described herein and an effective amount of NK cells, may be administered to a subject who needs treatment via a suitable route (e.g., intravenous infusion of the NK cells and/oral administration of the CTX compound). The cyclophosphamide compound and/or NK cells may be mixed with a pharmaceutically acceptable carrier to form a pharmaceutical composition prior to administration, which is also within the scope of the present disclosure. As mentioned above, the NK cells may be expanded ex vivo (e.g., exposed to a cytokine, including but not limited IL-12 and IL-15).

The NK cells may be autologous to the subject, i.e., the NK cells are obtained from the subject in need of the treatment. Administration of autologous cells to a subject may result in reduced rejection of the NK cells as compared to administration of non-autologous cells. Alternatively, the NK cells can be allogenic cells, i.e., the cells are obtained from a first subject, optionally exposed to cytokines (e.g., IL-12 and IL-15) and administered to a second subject that is different from the first subject but of the same species. For example, allogenic NK cells may be derived from a human donor and administered to a human recipient who is different from the donor. Alternatively, the NK cells may be derived from in vitro cell culture as described herein.

The subject to be treated may be a mammal (e.g., human, mouse pig, cow, rat, dog, rabbit, goat, sheep, or monkey). The subject may have, be suspected of having or be at risk for cancer. Exemplary cancers include solid tumors (e.g. breast cancer) and hematologic cancers. The type of breast cancer may be triple negative breast cancer. Exemplary hematologic cancers include but are not limited to leukemia, lymphoma (e.g., Non-Hodgkin lymphoma and Hodgkin lymphoma), Burkitt's lymphoma, chronic lymphocytic leukemia (CLL), chronic myelocytic leukemia (CML), acute myeloid leukemia (AML), acute lymphocytic leukemia (ALL), t-cell lymphoma (mycosis fungoides), and multiple myeloma. Exemplary solid tumors include, but are not limited to, neuroblastoma, retinoblastoma, breast cancer, and ovarian cancer. In some instances, the subject to be treated has cancer and has been previously treated for the cancer. For example, the subject may have had one or more tumors removed (i.e.: resected).

In some embodiments, the subject may be a human cancer patient who has undergone a prior anti-cancer therapy. Non-limiting examples include chemotherapy, immunotherapy, radiotherapy, surgery, or the combined therapy disclosed herein. The prior anti-cancer therapy may be complete. Alternatively, the prior anti-cancer therapy may still be on-going. In some embodiments, the human patient may exhibit tumor remission (e.g., complete or partial) after the prior therapy.

A cyclophosphamide compound and NK cells may be administered sequentially (in any order) to a subject. As used herein, the term “combination therapy” includes, inter alia, sequential administration of the referenced entities (e.g., NK cells and cyclophosphamide compounds). For example, a cyclophosphamide compound may be administered (e.g., at least 6 hours, at least 12 hours, at least 1 day, at least 3 days, at least 5 days, or at least 7 days) prior to the administration of NK cells. More than one dose (at least 2, at least 3, at least 4, at least 5, or at least 10 doses) of a cyclophosphamide compound may be administered to a subject. Similarly, More than one dose (at least 2, at least 3, at least 4, at least 5, or at least 10 doses) of NK cells may be administered to a subject. Alternating administration of a cyclophosphamide compound and NK cells is also encompassed by the present disclosure.

The amount of the CTX compound to be administered to a subject may depend on many factors, including the subject's height and weight, general health or other health problems, and the type of cancer or condition the subject has. The exact dosage and schedule may be determined by a physician. A CTX compound may be given to a patient via various routes, depending upon the dosage, the condition being treated, as well as the purpose it is being used for. For example, the CTX compound may be injected intravenous (intravenous, IV) or by oral administration (e.g., in tablet form), optionally after meals. Alternatively, a CTX compound may be given to a subject by intramuscular injection (IM), or injection to the abdominal lining (intraperitoneal, IP), or into the lining of the lung (intrapleural).

The NK cells may be administered to the subject, once or multiple times, via suitable route, for example, intravenous infusion. In some instances, a subject (e.g., a human patient) can be treated by infusing therapeutically effective doses of NK cells in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg.

Administration of a cyclophosphamide compound and NK cells to a subject may prevent tumor growth, inhibit tumor growth and/or induce tumor regression. For example, administration of a cyclophosphamide compound and NK cells to a subject may reduce the size of a tumor (e.g., volume of a tumor) by at least 10% (e.g., at least 20%, at least 30%, at least 40%, at least 50%, at least 60%, at least 80%, or at least 90%).

Subjects receiving combination treatment may survive longer than subjects not receiving a cyclophosphamide compound and NK cells. The combination of a cyclophosphamide compound and NK cells may result in a hazard ratio of less than 1 compared to no treatment. For example, the hazard ratio of the combination may be less than 0.9, 0.8, 0.7, 0.6, 0.5, 0.4, 0.3, 0.2, or 0.1. Non-limiting examples of hazard ratio measurements are provided in Table 1 and Table 3.

In some instances, administration of a cyclophosphamide compound and NK cells may induce adaptive immunity against tumor recurrence. For example, administration of a cyclophosphamide compound and NK cells may induce adaptive immunity against a specific type of tumor (e.g., solid tumors such as breast cancer, prostate cancer, liver cancer, lung cancer, melanoma, or pancreatic cancer, or a hematologic cancer such as those known in the art and/or disclosed herein). Treatment with a cyclophosphamide compound and NK cells may lower the risk for cancer recurrence. For example, combination therapy with cyclophosphamide compound and NK cell may increase the probability of disease-free survival (e.g., 5-year disease-free probability or 10-year disease-free survival probability). The probability of disease-free survival may be increased by at least 10% (e.g., by at least 20%, at least 30%, at least 40%, at least 50%, at least 60%, at least 70%, at least 80%, or at least 90%).

The CTX compounds and NK cells described herein may be utilized in conjunction with other types of therapy for cancer, including chemotherapy, surgery, radiation, gene therapy, targeted agents and so forth. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In some instances, a cyclophosphamide compound and NK cells are administered before tumor resection (e.g., in a subject with breast cancer). In some cases, a cyclophosphamide compound and NK cells are administered after tumor resection.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

Kits for Therapeutic Uses

The present disclosure also provides kits for use in any cancer described herein. A kit for therapeutic use as described herein may include one or more containers comprising (i) a cyclophosphamide compound (e.g., CTX), and (ii) NK cells, which may have been cultivated ex vivo with a cytokine (e.g. IL-12 and IL-15). The cyclophosphamide compound and/or NK cells may be formulated in a pharmaceutically acceptable carrier. The kit may further comprise IL-12 and IL-15.

In some embodiments, the kit can additionally comprise instructions for use of cyclophosphamide compound and NK cells in any of the methods described herein. The included instructions may comprise a description of administration of the NK cells, the cyclophosphamide compound, or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the cyclophosphamide compound, the NK cells, or the pharmaceutical composition comprising such to a subject who is in need of the treatment.

The instructions relating to the use of the cyclophosphamide compound, the NK cells, or the pharmaceutical composition comprising such as described herein generally include information as to dosage, dosing schedule, and route of administration for the intended treatment. The containers may be unit doses, bulk packages (e.g., multi-dose packages) or subunit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. NK cells and a cyclophosphamide compound may be considered active agents.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Therapeutic Effect of CTX Treatment on EO771 Tumor-Bearing Mice

The syngeneic orthotopic EO771 breast cancer mouse model (see Johnstone et al., Disease models & mechanisms, 8(3):237-251 (2015)) was used to evaluate the in vivo treatment efficacy.

Materials and Methods

Mice

C57BL/6JNarl female mice were purchased from National Laboratory Animal Center, Taiwan and housed under specific pathogenic-free condition in the animal facility of Institute of Molecular Biology, Academia Sinica, Taiwan. Mice were used between 8-to-12-week-old.

Cell Line and Cell Culture

EO771 cell line (CH3 BioSystems) were cultured in complete medium (RPMI 1640 (Gibco) supplemented with 20 mM pH7.2 HEPES (Sigma-Aldrich), 10% fetal bovine serum (FBS; HyClone) and 1% Penicillin and Streptomycin (PS; Gibco)). B16-F10 melanoma was provided by Dr. Roffler, Steve R. (Institute of Biomedical Sciences, Academia Sinica, Taiwan) and cultured in complete medium (DMEM (Gibco) supplemented with 10% FBS (HyClone) and 1% PS (Gibco)). Tumor cells were cultured at 37° C. incubator with 5% CO₂.

Preparation of Murine NK Cells

Bone marrow (BM) cells were obtained from Tibias and femurs. Red blood cells were lysed by ACK lysis buffer (0.15 M NH4Cl, 10 mM NaHCO₃, 1 mM disodium EDTA (pH 7.4)). The BM cells were cultured in 10% FBS RPMI medium containing rmIL-15 for 7 days, and IL-12 was added 16 hours before harvesting. The harvested BM cells were stained with antibodies specific for CD19, H57, NK1.1, CD11 c and B220, and sorted for CD11c⁺B220⁺ NK cells using FACSAriaII SORP (BD Biosciences; FACS Core, Institute of Molecular Biology, Academia Sinica, Taiwan).

Tumor Models and Treatment Regimen

EO771 cell line derived from a spontaneous medullary breast adenocarcinoma of C57BL/6 mouse. The orthotopic syngeneic TMBC model was done by injection of EO771 cells into the 4th mammary fat pad of C57BL/6JNarl female mice. For the tumor-bearing model, each mouse was inoculated with 0.5×10⁶ cells and received the first intra-peritoneal injection of CTX after 21 days. The second CTX injection was given 6 days later. Sorted CD11c⁺B220⁺ NK cells were transferred intravenously around 24 hours after the second CTX treatment, and followed with another NK cell transfer 3-4 days later. For the tumor-resect model, each mouse was inoculated with 0.3×10⁶ EO771 cells, and the tumor and draining lymph node were resected 21 days later. CTX/NK treatment started at 48 hours after resection as described in the tumor-bearing model. B16-F10 melanoma cells (0.075×10⁶ cells/mouse) were inoculated subcutaneously for re-challenge as indicated. The mice were monitored for survival, body weight, and tumor volume.

Statistical Analysis

Kaplan-Meier survival analyses were performed using GraphPad Prism 7, and the Log-rank test was used to determine significance. Cox proportional hazard model was used to analyze hazard ratio and interaction [24]. P-value≤0.05 was considered statistically significant (*p≤0.05, **p≤0.01, ***p≤0.001, ****p≤0.0001).

Results

To develop an effective regimen, the effect of once versus twice cyclophosphamide (CTX) treatments on tumor-bearing mice was investigated. CTX was administrated on day-21 post tumor inoculation. The twice CTX-treatment group received the second CTX injection 6 days after the first treatment. Phosphate-buffered saline (PBS) was used as a negative control.

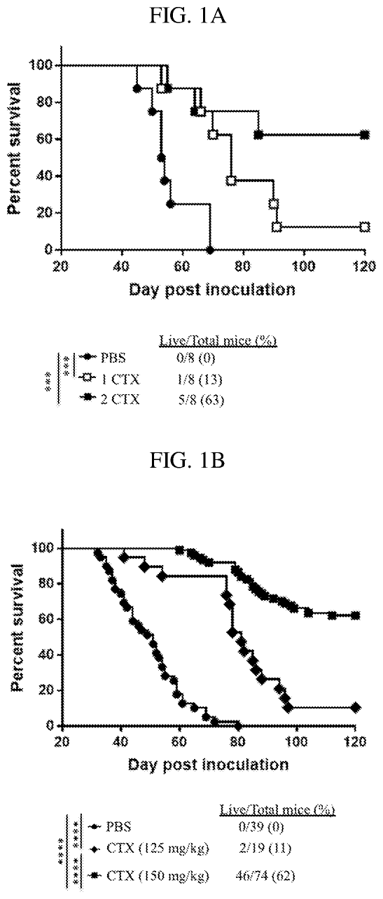

All mice received phosphate-buffered saline (PBS) died with a median survival of 53 days. On the other hand, 1 out of 8 mice treated with CTX once and 5 out of 8 mice treated with CTX twice survived for at least 120 days. FIG. 1A. The results indicate that CTX is effective in treating breast cancer and multiple administration (e.g., with a 6-day interval) showed better results.

The two-administration (6-day apart) CTX treatment regimen was used in the following experiments, using 125 mg/kg and 150 mg/kg per injection CTX dosage. It was found that the survival rate was significantly improved from 11% to 62% by the 150 mg/kg dosage. (FIG. 1B). Based on these results, twice CTX treatment at the dosage of 150 mg/kg/injection delivered 6 days apart was used to test combination therapy with adoptive NK cell transfer.

### Example 2: CTX and NK Cell Therapies Synergistically Enhance the Survival of EO771 Tumor-Bearing Mice

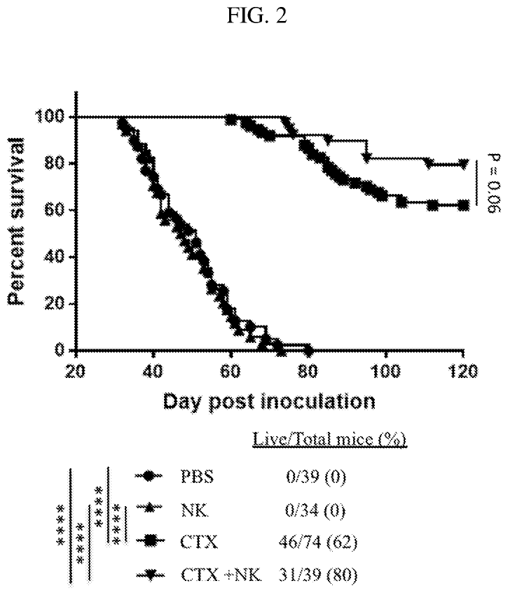

First, the effects of CTX monotherapy, NK monotherapy, or CTX and NK cell combined therapy on EO771 tumor-bearing mice were examined, following the assays described in Example 1 above. The treatment started on day-21 post tumor inoculation. All mice received NK cell monotherapy died with a median survival rate similar to that of the negative control (PBS) group. FIG. 2. On the other hand, 62% of mice in the CTX monotherapy group and 80% of mice in the CTX and NK cell combined therapy group survived for at least 120 days. FIG. 2.

Analyses with Cox proportional hazards model indicated significantly lower hazard ratio for CTX monotherapy and CTX and NK cell combined therapy, as compared with the PBS control group. A significant benefit was observed in the combined therapy group as compared with the CTX monotherapy. The results are shown in Table 1 below. Moreover, the CTX and NK cell combined therapy showed a multiplicative interaction with a hazard ratio of 0.353 (p=0.028), indicating the synergistic effect of CTX and NK cell in achieving long-term survival effect for treatment of tumor-bearing mice as exemplified in the EO771 model.

TABLE 1
Hazard ratio (HR) calculations for CTX, NK, and CTX plus NK cell therapies
compared with no treatment in EO771 tumor-bearing mice.

| Tumor-bearing | Without batch adjustment | | Frailty adjustment | | Cluster adjustment | |
|---|---|---|---|---|---|---|
| model Treatment | HR (95% CI) | p-value | HR (95% CI) | p-value | HR (95% CI) | p-value |
| No treatment | reference | — | reference | — | reference | — |
| CTX | 0.024 (0.012, 0.051) | <0.001 | 0.02 (0.009, 0.044) | <0.001 | 0.024 (0.009, 0.066) | <0.001 |
| NK | 1.362 (0.829, 2.240) | 0.223 | 1.209(0.605, 2.414) | 0.59 | 1.162 (0.644, 2.881) | 0.419 |
| CTX + NK | 0.012 (0.005, 0.031) | <0.001 | 0.009(0.003, 0.024) | <0.001 | 0.012 (0.005, 0.029) | <0.001 |
| CTX | reference | — | reference | — | reference | — |
| CTX + NK | 0.481 (0.219, 1.055) | 0.0677 | 0.445 (0.201, 0.986) | 0.0462 | 0.481 (0.284, 0.814) | 0.00644 |

Therefore, the CTX/NK cell combined therapy showed significant benefit as compared with the CTX monotherapy under the tumor-bearing conditions. CTX and NK cell combined therapies showed multiplication interaction in the tumor-bearing mice, indicating synergy between the two therapeutic agents. Furthermore, CTX monotherapy and CTX/NK cell combined therapies, but not the NK cell monotherapy, promoted survival of tumor-bearing mice.

### Example 3. CTX and NK Cell Combined Therapy Induces Protective Immune Memory in EO771 Tumor-Bearing Mice

Cancer recurrence is a major problem in cancer therapy. To determine whether CTX/NK cell combined therapy would prevent or reduce the risk of tumor recurrence, mice who survived the primary tumor after the treatment were re-challenged with the same tumor cells to mimic cancer recurrence. The assays used in this Example are provided in Example 1 above.

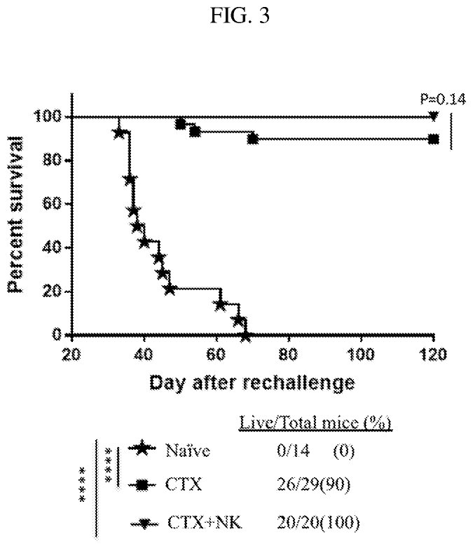

It was found that 100% of the CTX/NK cell combined therapy group and 90% of the CTX monotherapy group survived at least 120 days after the EO771 re-challenge, while all age-matched naive mice inoculated with EO771 cells died up to 70 days after re-challenge. FIG. 3. These results suggested that treatment of primary tumor with both CTX and NK cells induces immune memory that protects the mice from tumor recurrence.

In the tumor-bearing model, the CTX and NK cell combined therapy showed higher efficacy than the CTX monotherapy in primary survival investigation, while they induced comparable protection against re-challenge with the same tumor cells. The cure rate was estimated by multiplying the primary and re-challenge survival rates. As shown in Table 2 below, the cure rate was 80% for the CTX/NK cell combined therapy and 56% for the CTX mono-therapy.

TABLE 2
Primary survival rate, survival rate after E0771
re-challenge and estimated cure rate with immune
protection in tumor-bearing mice following treatment.

| | | Survival rate | |
|---|---|---|---|
| Tumor-bearing | Primary | after EO771 | Estimated cure |
| mice | survival rate¹ | re-challenge | rate with immune |
| Treatment | % (live/total) | % (live/total) | protection² (%) |
| NK | 0 (0/34) | not applicable | not applicable |
| CTX | 62 (46/74) | 90 (26/29) | 56 |
| CTX + NK | 80 (31/39) | 100 (20/20) | 80 |

¹Survival rate post treatment in mice inoculated with tumor the first time.
²Estimated cure rate with immune protection: Primary survival rate × Survival rate after EO771 re-challenge.

Taken together, adoptive NK cell transfer and CTX combined therapy showed synergistic anti-tumor effect on EO771 tumor-bearing mice, and reached an estimated cure rate of 83%. The results also suggest that the CTX/NK cell combined therapy and the CTX monotherapy would induce immune memory protecting against tumor recurrence under tumor-bearing conditions.

### Example 4. CTX Monotherapy and CTX/NK Cell Combined Therapy Achieved Comparable Efficacy in the Survival of EO771 Tumor-Resected Mice

Next, the effect of CTX monotherapy and CTX/NK cell combined therapy were examined in an EO771 tumor resection mouse model, which mimics patients whose tumor was surgically removed. The assays used in this Example are provided in Example 1 above.

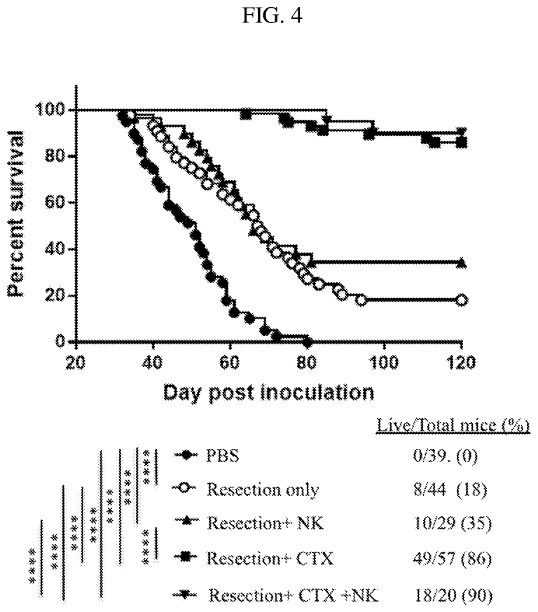

Tumor and draining lymph node were resected on day-21 post tumor inoculation, and CTX alone or in combination with NK cells were started two days after the resection. Mice received neither resection nor treatment (PBS control group) died with a median survival of 51 days, and resection alone resulted in an 18% survival with a median survival of 68 days. FIG. 4. In tumor resected mice, NK cell and CTX mono-therapies improved the survival rate to 35% and 86%, respectively. FIG. 4. CTX and NK cell combined therapy did not further enhance the survival rate compared with CTX alone. FIG. 4. Analyses with Cox proportional hazards model indicate significantly lower hazard ratio for the CTX monotherapy and the CTX/NK cell combined therapy groups as compared with the resection only group, while the CTX monotherapy and the CTX/NK cell combined therapy did not show significant difference. Table 3.

TABLE 3
Hazard ratio (HR) calculations for the CTX, NK, and CTX plus NK cell groups
compared with the resection only group in EO771 tumor-resected mice.

| Tumor-resection | without batch adjustment | | Frailty adjustment | | cluster adjustment | |
|---|---|---|---|---|---|---|
| model Treatment | HR (95% CI) | p-value | HR (95% CI) | p-value | HR (95% CI) | p-value |
| no treatment | reference | — | reference | — | reference | — |
| CTX | 0.086 (0.040, 0.186) | <0.001 | 0.083 (0.036, 0.192) | <0.001 | 0.086 (0.037, 0.201) | <0.001 |
| NK | 0.706 (0.405, 1.233) | 0.222 | 0.7 (0.354, 1.385) | 0.31 | 0.706 (0.497, 1.042) | 0.0799 |
| CTX + NK | 0.058 (0.014, 0.244) | <0.001 | 0.061 (0.014, 0.270) | <0.001 | 0.058 (0.014, 0.242) | <0.001 |
| CTX | reference | — | reference | — | reference | — |
| CTX + NK | 0.682 (0.145, 3.210) | 0.628 | 0.732 (0.150, 3.587) | 0.701 | 0.682 (0.127, 3.656) | 0.655 |

In sum, CTX alone and CTX and NK cell in combination promoted survival of tumor-resected mice. These results indicate that CTX mono-therapy and CTX/NK cell combined therapy achieved comparable high efficacy in the primary survival of tumor-resected mice.

### Example 5. NK Cell Treatment Potentiates Tumor-Specific Immune Memory in EO771 Tumor-Resected Mice

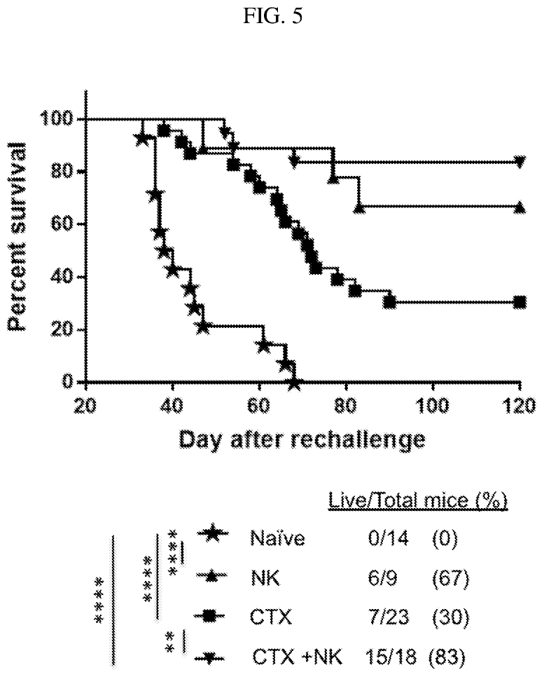

Following the assays described in Example 1 above, it was examined in this Example whether CTX monotherapy and CTX/NK cell combined therapy would induce protective immune memory under tumor-resected condition. Mice survived after the treatment of primary EO771 tumor were re-challenged with EO771 cells. The CTX group showed a 30% survival, while the NK cell group showed a 67% survival. FIG. 5. Combination of CTX and NK cell showed the highest protection of 83% survival. FIG. 5. On the other hand, all mice re-challenged with B16F10 died despite of the treatment type for the primary EO771 tumor. Base on the EO771 primary and re-challenge survival rates, the estimated cure rate for tumor-resected mice was 23%, 26%, and 75% for NK, CTX, and CTX+NK therapies, respectively. Table 4 below.

TABLE 4
Primary survival rate, tumor-free rate after E0771
re-challenge, and estimated cure rate with immune
protection in EO771 tumor-resected mice following treatment.

| Tumor-resected | Primary | Tumor-free rate after | Estimated cure |
|---|---|---|---|
| mice | survival rate¹ | EO771 re-challenge | rate with immune |
| Treatment | % (live/total) | % (live/total) | protection² (%) |
| NK | 35 (10/29) | 67 (6/9) | 23 |
| CTX | 86 (49/57) | 30 (7/23) | 26 |
| CTX + NK | 90 (18/20) | 83 (15/18) | 75 |

¹Survival rate post treatment in mice inoculated with tumor the first time.
²Estimated cure rate with immune protection: Primary survival rate × Survival rate after EO771 re-challenge.

Taken together, the CTX and NK cell combined therapy achieved an estimated cure rate of 75%, which is significantly higher than the corresponding monotherapies. CTX and NK cell combined therapy induced tumor-specific protection against recurrence under tumor-resected conditions.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of treating cancer, comprising:
a. administering to a subject in need thereof an effective amount of a cyclophosphamide (CTX) compound; and
b. administering to the subject an effective amount of natural killer (NK) cells;
wherein the cancer is a solid cancer or a hematologic cancer; and
wherein the CTX compound in step (a) is administered to the subject before step (b).

2. The method of claim 1, wherein the CTX compound is cyclophosphamide, mafosfamide, ifosfamide, or trofosfamide, or a pharmaceutically acceptable salt thereof.

3. The method of claim 1, wherein step (a) comprises at least two doses of the CTX compound and/or step (b) comprises at least two doses of the NK cells.

4. The method of claim 3, wherein step (a) comprises at least two doses of the CTX compound, and wherein administration of two consecutive doses of the CTX compound is at least 4-8 days apart.

5. (canceled)

6. (canceled)

7. The method of claim 1, wherein the subject is a human patient having, suspected of having, or at risk for cancer.

8. The method of claim 7, wherein the solid cancer is selected from the group consisting of breast cancer, prostate cancer, liver cancer, lung cancer, melanoma, pancreatic cancer, and bladder cancer, or the hematologic cancer is selected from the group consisting of leukemia, lymphoma, and multiple myeloma.

9-11. (canceled)

12. The method of claim 1, wherein the subject has had a tumor resection.

13. The method of claim 1, wherein the effective amount of the CTX compound and the effective amount of NK cells are effective in reducing the risk of cancer recurrence.

14. The method of claim 1, wherein the NK cells have been exposed to IL-15 and IL-12 ex vivo prior to step (b).

15. A method of inducing protective immune memory against tumor recurrence and/or reducing the risk of tumor recurrence, the method comprising:
a. administering to a subject in need thereof an effective amount of a cyclophosphamide (CTX) compound; and
b. administering to the subject an effective amount of natural killer (NK) cells;
wherein the tumor is a solid cancer or a hematologic cancer; and
wherein the CTX compound in step (a) is administered to the subject before step (b).

16. The method of claim 15, wherein the CTX compound is cyclophosphamide, mafosfamide, ifosfamide, or trofosfamide, or a pharmaceutically acceptable salt thereof.

17. The method of claim 15, wherein the subject is a human patient who had tumor.

18. The method of claim 17, wherein the human patient has undergone an anti-tumor therapy.

19. The method of claim 15, wherein the NK cells are autologous or allogenic.

20. The method of claim 15, wherein the NK cells are from an NK cell line, derived from pluripotent stem cells, or derived from induced-pluripotent stem cells.

21. A kit for use in treating cancer, comprising (i) a cyclophosphamide (CTX) compound; and (ii) natural killer (NK) cells;
wherein the cancer is a solid cancer or a hematologic cancer; and
wherein the CTX compound is administered to the subject before administration of the NK cells.

22. The kit of claim 21, wherein the NK cells have been exposed to IL-15 and IL-12 ex vivo.

23. The kit of claim 21, wherein the CTX compound is cyclophosphamide, mafosfamide, ifosfamide, or trofosfamide, or a pharmaceutically acceptable salt thereof.
